(19) United States
(12) Patent Application Publication
(10) Pub. No.: US 2022/0343404 A1
(43) Pub. Date: Oct. 27, 2022

(54) METHOD AND SYSTEM FOR PREDICTIVE REAL-TIME FOOT TRAFFIC ANALYTICS

(71) Applicant: Andie Connected Technologies Inc., Toronto (CA)

(72) Inventor: Baba Ajayi, Toronto (CA)

(21) Appl. No.: 17/238,347

(22) Filed: Apr. 23, 2021

(51) Int. Cl. G06Q 30/06; G06Q 10/10; G16H 50/80

(52) U.S. Cl. CPC G06Q 30/0641; G06Q 10/1095; G16H 50/80

(57) ABSTRACT

Methods and systems for predictive, real-time foot traffic and contact tracing analytics are provided. According to one example, a server maintains a database of foot traffic signals related to a plurality of stores, receives a plurality of foot traffic signals associated with the plurality of stores, synthesizes the plurality of foot traffic signals, and updates the database using the synthesized plurality of foot traffic signals. Upon receiving a data query from a user electronic device including a location data parameter, the server generates a foot traffic prediction based on the data query and the location data parameter from the database and forwards a foot traffic prediction notification for display on the user electronic device.

### FIELD

The field of the invention is data analytics and digital communications and, in particular, a method and system for predictive, real-time foot traffic and contact tracing analytics.

### BACKGROUND

The following description includes information that may be useful in understanding the present invention. It is not an admission that any of the information provided herein is prior art or relevant to the presently claimed invention, or that any publication specifically or implicitly referenced is prior art.

Foot traffic, also known as footfall or shopper counting refers to the measurement of the number of people entering or queueing for a shop or shopping mall. Predicting foot traffic is a challenging problem that has been exacerbated by the global pandemic of the Coronavirus disease (COVID-19), an infectious disease caused by a newly discovered coronavirus. Generally speaking, the virus that causes COVID-19 is transmitted when an infected person coughs, sneezes, or exhales. In an effort to prevent the spread of infection, countries and territories around the world have implemented curfews, quarantines, and similar restrictions (sometimes described as stay-at-home orders, shelter-in-place orders, shutdowns, lockdowns, emergency brake measures, and the like). These restrictions have affected hospitality establishments, retailers, gyms, grooming and other venues, among many other service providers and locations where people assemble.

In cases where retailers or other service providers are allowed to open, they can be subject to corporate, landlord or government enforced rules such as limits on store occupancy, curb-side pickup requirements, outdoor dining requirements, personal protective equipment requirements, mandatory screening of customers and employees, contact tracing requirements, and special rules for returns, fitting rooms, bagging, etc. The trajectory of the disease in different locations (based on measures of cases of infections, hospital capacity, death rates, vaccination rates, etc.) means that restrictions can be phased in or out over time, are not necessarily uniform over time and in all locations, and can be different depending on the service offered, throwing up a further obstacle to accurate foot traffic predictions.

Retail trends such as consumer preferences away from bricks-and-mortar shopping to e-commerce or hybrid models and the wide availability of delivery services have also made predicting foot traffic more challenging.

For retailers or other service providers, more accurate and timely prediction of foot traffic can help with decisions as to inventory, staffing levels, and operating hours, and to ensure compliance with local rules and regulations which as noted above are subject to change frequently. Consumers also benefit from analytics drawn from more accurate foot traffic predictions when making decisions around where and when to shop, and how much time to expect in a queue or a store at a given location at a particular time. Previous approaches that used past information may not necessarily provide accurate predictions about the present, and could not provide updated predictions in real-time.

In public health, contact tracing is the process of identifying persons who may have come into contact with an infected person and subsequent collection of further information. The goals of contact tracing may include one or more of the following: to interrupt ongoing transmission and reduce the spread of an infection; to alert contacts to the possibility of infection and offer preventive services or prophylactic care; to offer diagnosis, counselling and treatment to already infected individuals; if the infection is treatable, to help prevent reinfection of the originally infected patient; to learn about the epidemiology of a disease in a particular population. Systems have been developed based on Bluetooth Low Energy (BLE) wireless radio signals for proximity information, with apps or services on Apple and Google devices warning users that they had been in contact with an infected person. Existing systems suffer from drawbacks including that not all patrons of a retailer or hospitality establishment may carry a device. Furthermore, rights to medical confidentiality present an ongoing challenge with any computer-implemented contact tracing efforts.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of features used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Thus, there is a need for a system and a method that allows for foot traffic and contract tracing analytics to mitigate, or at least to provide an alternative way to address, some of the obstacles to effective and secure prediction of retail measures such as foot traffic and/or contact tracing.

### BRIEF DESCRIPTION OF THE DRAWINGS

Various objects, features, aspects and advantages of the inventive subject matter will become more apparent from the following detailed description of preferred embodiments, along with the accompanying drawing figures in which like numerals represent like components.

FIG. 1 is a block diagram of a system architecture for predictive real-time foot traffic and contact tracing analytics in accordance with an example of the present specification;

FIG. 2 is a block diagram of a server for predictive real-time foot traffic and contact tracing analytics according to the system architecture of FIG. 1 in accordance with an example;

FIG. 3 is a block diagram of a client device for predictive real-time foot traffic and contact tracing analytics according to the system architecture of FIG. 1 in accordance with an example;

FIG. 4 is a flowchart of a method of generating a real-time foot traffic prediction, in accordance with an example of the present specification; and

FIG. 5 and FIG. 6 are views illustrating client application screenshots, in accordance with examples of the present disclosure.

### DETAILED DESCRIPTION

Throughout the following discussion, numerous references may be made regarding servers, services, interfaces, engines, modules, clients, peers, portals, platforms, or other systems formed from computing devices. It should be appreciated that the use of such terms is deemed to represent one or more computing devices having at least one processor (e.g., ASIC, FPGA, DSP, x86, ARM, ColdFire, GPU, multi-core processors, etc.) configured to execute software instructions stored on a computer readable tangible, non-transitory medium (e.g., hard drive, solid state drive, RAM, flash, ROM, etc.). For example, a server can include one or more computers operating as a web server, database server, or other type of computer server in a manner to fulfill described roles, responsibilities, or functions. One should further appreciate the disclosed algorithms, processes, methods, or other types of instruction sets can be embodied as a computer program product comprising a non-transitory, tangible computer readable media storing the instructions that cause a processor to execute the disclosed steps. The various servers, systems, databases, or interfaces can exchange data using standardized protocols or algorithms, possibly based on HTTP, HTTPS, AES, public-private key exchanges, web service APIs, known protocols, or other electronic information exchanging methods. Data exchanges can be conducted over a packet-switched network, the Internet, LAN, WAN, VPN, or other type of packet switched network.

One should appreciate that the systems and methods of the inventive subject matter provide various technical effects, including providing predictive real-time foot traffic and contact tracing analytics.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In this specification, methods and systems for predictive, real-time foot traffic and contact tracing analytics are provided.

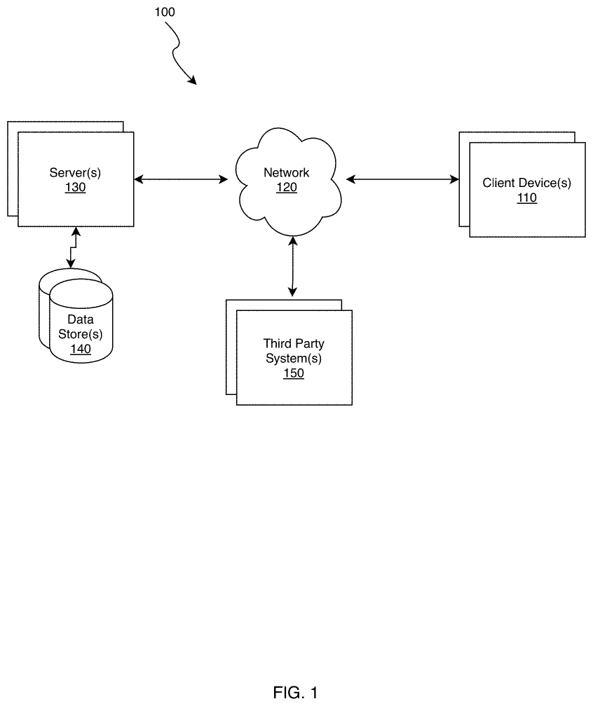

FIG. 1 illustrates an example architecture of the system 100. System 100 includes one or more client devices 110, one or more servers 130 (linked to one or more data stores 140), and one or more third party systems 150 connected to each other by a network 120. Although FIG. 1 illustrates a particular arrangement of client devices 110, servers 130, data stores 140, third party systems 150, and network 120, the present specification contemplates any suitable arrangement of these components. As an example and not by way of limitation, two or more of client devices 110, servers 130, data stores 140, and third party systems 150 may be connected to each other directly, bypassing network 120. As another example, the functions of client device 110 and/or server 130, data store 140 and/or third party system 150 may be combined in a single device or service. The present specification contemplates any suitable number of client devices 110, servers 130, data stores 140, third party systems 150, and networks 120.

The present specification encompasses any suitable network 120. As an example and not by way of limitation, one or more portions of network 120 may include an ad hoc network, an intranet, an extranet, a virtual private network (VPN), a local area network (LAN), a wireless LAN (WLAN), a wide area network (WAN), a wireless WAN (WWAN), a metropolitan area network (MAN), a portion of the Internet, a portion of the Public Switched Telephone Network (PSTN), a cellular telephone network, or a combination of two or more of these. Network 120 may include one or more networks 120.

The lines connecting components of the system 100 are called links. The present specification contemplates any suitable links that may include one or more wireline, wireless, or optical links. One or more links can each include an ad hoc network, an intranet, an extranet a VPN, a LAN, a WLAN, a WAN, a WWAN, a MAN, a portion of the Internet, a portion of the PSTN a cellular technology-based network, a satellite communications technology-based network, another link, or a combination of two or more such links. The links need not necessarily be the same throughout the system 100.

In particular embodiments, a third party system 150 may include one or more types of servers, one or more data stores, one or more interfaces, including but not limited to APIs, one or more web services, one or more content sources, one or more networks, or any other suitable components. A third party system 150 may be operated by a different entity from an entity operating the server 130. In particular embodiments, server 130 and third party systems 150 may operate in conjunction with each other to provide inventory, analytics, appointment or other communication services to users of server 130 or third-party systems 150. In this sense, server 130 may provide a platform, or backbone, which other systems, such as third party systems 150, may use to provide services and functionality to users across the Internet

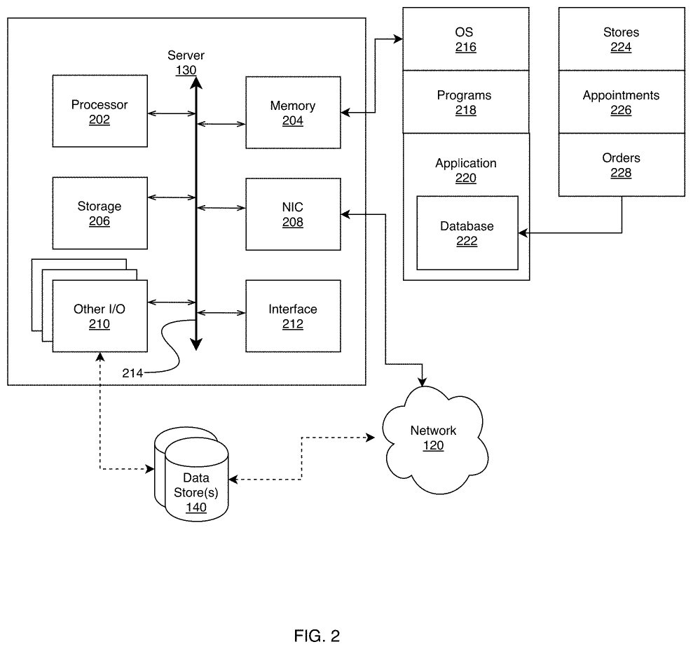

Turning now to FIG. 2, a block diagram of an example of a server 130 for providing predictive, real-time foot traffic and contact tracing analytics, is shown. The server 130 is typically a server or mainframe within a housing containing an arrangement of one or more processors 202, volatile memory (i.e., random access memory or RAM) (shown as memory 204), persistent memory (e.g., hard disk or solid state devices) (shown as storage 206), and a network interface controller 208 (to allow the server 130 to communicate over the network 120) interconnected by a bus 214. Many computing environments implementing the server 130 or components thereof are within the scope of the present specification. The server 130 may include a pair (or more) of servers for redundancy or load-balancing purposes, connected via the network 120 (e.g., an intranet or across the Internet). The server 130 may be connected to other computing infrastructure including displays, printers, data warehouse or file servers, and the like. The server 130 may include a keyboard, mouse, touch-sensitive display (or other input devices), a monitor (or display, such as a touch-sensitive display, or other output devices) (shown generically as I/O devices 210 in FIG. 2).

As noted above, server 130 includes a network interface controller or NIC 208 interconnected with the processor 202 that allows the server 130 to communicate with other computing devices such as one or more client devices 110 via a link with the network 120. As indicated, network 120 may include any suitable combination of wired and/or wireless networks, including but not limited to a Wide Area Network (WAN) such as the Internet, a Local Area Network (LAN), HSPA/EVDO/LTE/5G cell phone networks, Wi-Fi networks, and the like. The NIC 208 is selected for compatibility with the network 120. In one example, the link between the NIC 208 and the network is a wired link, such as an Ethernet link. The NIC 208 thus includes the necessary hardware for communicating over such a link. In other examples, the link between the server 130 and the network 120 may be wireless, and the NIC 208 may include (in addition to, or instead of, any wired-link hardware) one or more transmitter/receiver assemblies, or radios, and associated circuitry.

Typically, the server 130 may be coupled to the data store 140 over a bus or a network (such as network 120) (links shown in dotted lines in FIG. 2) and the server 130 may access or cache data from the data store 140 at run-time, or at predetermined times, using an API (application program interface).

In the above noted example, the server 130 maintains one or more data stores 140. The data stores 140 maintain foot traffic signal data, as will be explained in more detail below.

The server 130 includes an operating system 216 and software programs 218, applications, or components (not shown) that are executed by the processor and are typically stored in a persistent, updatable store such as the data storage 206. Additional applications or programs may be loaded onto the server 130 through the network 120 or any other I/O devices 210 or subsystem. The server 130 includes an application 220 that provides access to a database 222 loaded on the server 130 that can be a stand-alone database server (shown as data store 140) or a virtual machine in communication with the NIC 208 of the server 130, or any other suitable database. The database 222 maintains data records associated with stores 224, data records associated with appointments 226 and data records associated with orders 228.

In one example, the server 130 may be integral with the client device 110. According to this example, at least some of the data records from the database 222 may be maintained directly on the client device 110, permitting use in an “offline” modality.

An interface 212 of the server 130 can access data within the data store 140 and/or the storage 206 such as the database 222. According to one example, interface 212 can comprise one or more computing devices that enables a user or data analyst to access data from the server 130, such as by through server application 220.

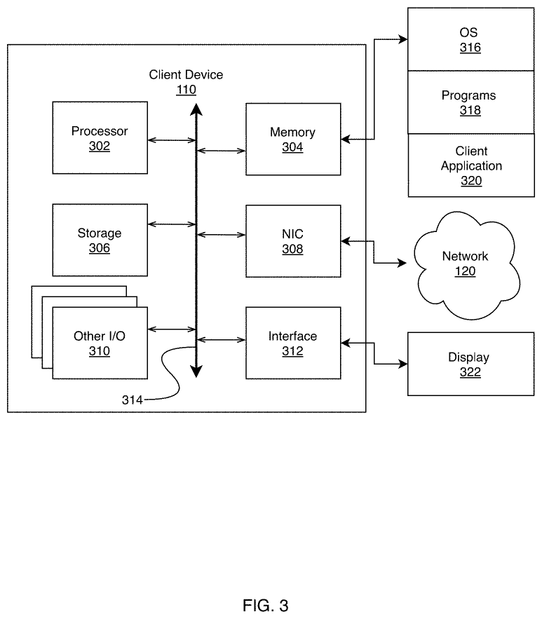

Turning to FIG. 3, in particular embodiments, client device 110 may be an electronic device including hardware, software or embedded logic components or a combination of two or more such components and capable of carrying out the appropriate functionalities implemented or supported by client device 110. As an example and no by way of limitation, a client device 110 may include a computer system such as a desktop computer, notebook or laptop computer, netbook, a tablet computer, e-book reader, GPS device, camera, personal digital assistant (PDA) handheld electronic device, cellular telephone, smartphone, smartwatch, smartgoggles, smart speaker, Internet of Things device, augmented/virtual reality device, or other suitable electronic device, or any suitable combination thereof. This disclosure contemplates any suitable client devices 110. A client device 110 may enable a user at client device 110 to access network 120.

According to one example, the client device 110 includes multiple components, such as a processor 302 that controls the overall operation of the client device 110. Communication functions, including data communications, are performed through a communication subsystem (shown as Network Interface Controller or NIC 308). The NIC 308 receives messages from and sends messages to a network 120. A power source (not shown), such as one or more rechargeable batteries or a port to an external power supply, powers the client device 110. The processor 302 of the client device 110 interacts with other components, such as a Random Access Memory (RAM) 304, data storage 306, a display 322, one or more speakers (not shown), a microphone (not shown), and one or more other sub-systems such as other I/O devices 310 including a short-range communications subsystem.

According to an example, input via a graphical user interface can be provided via the display 322 which may be a touch-sensitive display. Alternatively, according to a different example, input can be provided via elicitation using the microphone. Information, such as text, characters, symbols, images, icons, and other items that may be displayed or rendered on a client device 110, is displayed on the display 322 via the processor 302. The information can be displayed or rendered on one or more of the client devices 110. The display 322 may be any suitable touch-sensitive display, such as a capacitive, resistive, infrared, surface acoustic wave (SAW) touch-sensitive display, strain gauge, optical imaging, dispersive signal technology, acoustic pulse recognition, and so forth.

The client device 110 includes an operating system 316 and software programs 318, applications, or components (not shown) that are executed by the processor and are typically stored in a persistent, updatable store such as the data storage 306. Additional applications or programs may be loaded onto the client device 110 through the network 120, the short-range communications subsystem, or any other I/O devices or subsystem.

Those having ordinary skill in the related arts will readily appreciate that the preceding client device 110 and the server 130 are merely illustrative of the broader array of possible topologies and functions implementing systems of the present specification. Moreover, it should be recognized that various implementations can combine and/or further divide the various entities illustrated in FIG. 1, in FIG. 2, and in FIG. 3. For example, the server 130 can be stored or executed on one or more virtual machines. As is known in the art, a virtual machine is an execution environment (typically on a server) that has access to one or more processors, memory, disk drives, network interface cards, and so on. While one or more virtual machines can be instantiated on a single server, the processes of two different virtual machines typically do not interfere with one another (i.e., one virtual machine will not write over the data of another virtual machine, etc.) In the present case, one or more of the functions of the server 130 may be executed on a virtual machine, for example, provided by Amazon Web Services (AWS), Microsoft Azure, or another cloud service.

### Wait Time Predictions

According to particular embodiments, client application 320 of the client device 110 communicates with the server 130 to display information associated with foot traffic signals, such as wait times at a store, as well as other stores in a given proximity of the detected location of the client device 110. According to these embodiments, when the client application 320 is opened on the client device 110, a request to the server 200 triggers the server 200 to generate a predicted wait time for each store within a proximity based on a number of foot traffic signals including the time of day, the weather, historical data (e.g., how store traffic may be trending in a particular store within the previous week or other time interval), the size of the store, the capacity restrictions that may be established by the store or a local authority, the popularity of the store, the average income of persons in the neighbouring area, to name a few. According to one example, the client application 320 displays information regarding 10-50 stores within a 10 km radius of the user's location.

Use of the phrase foot traffic signal refers to a piece of data or a measurement associated with foot traffic in a store. Foot traffic signals can be collected by a people counter device which is an electronic device that is used to measure the number of people traversing a certain passage or entrance. Examples include simple manual clickers, smart-flooring technologies, infrared beams, thermal imaging systems, WiFi trackers and video counters using machine learning algorithms.

The term store refers to any retail establishment that sells items or provides services to the to the public. As used in the present specification, the term store extends not only to retailers but also to hospitality service providers such as hotels, bars and restaurants or indeed to any venue or establishment that is open to the public.

Foot traffic signals can also extend to the number of households in a given area, the proximity of the store to other stores of its kind, which stores other users are looking up and inquiring about the most using client applications 320 on their devices, if the store is offering any special privileges to any groups during that time of the day (e.g. seniors' hours), the wait time that users reported previously from that location for that particular time of the day, and what the weather is like that day and how it might influence people to go outside. The time of the day is also factored into the wait time analysis.

According to one example embodiment, foot traffic signals can be represented as factors or inputs into a weighted time-series algorithm. In this algorithm, a coefficient value for each factor is calculated. The coefficient is weighted to reflect the importance of that particular factor to the wait time that a user might face in the stores within a given proximity. Those numerical values are then inserted into an equation to calculate the predicted wait time. Once the wait time is calculated, the answer can then be cross-referenced to a time-series dataset containing previous wait times at the store location. The weighted time-series algorithm employed can be an algorithm selected from any of auto-regressive, moving average, autoregressive moving average, autoregressive integrated moving average, or exponential smoothing. Any method of time-series analysis may be employed without departing from the scope of the present specification.

Advantageously, by cross-referencing real-time factors with historical factors, the time-series algorithm can also identify potential cause for data outliers. For example, if the only other time a given store had a 50-minute wait time was when it snowed in April 2020, the system is able to identify this event.

According to particular embodiments, foot traffic signals can be inputted manually. Time and service speed information, such as opening hours, turn-around time targets for a range of goods and services can be specified directly by the retail users, providing an additional signals to generate wait time predictions. As well, search engine traffic can also be inputs or foot traffic signals. For example, the system can is able to leverage data from the number of people searching for a store's opening hours, wait times, etc. and or aspects of their queries (i.e, potential visitor inputs).

In particular embodiments, the number of users in a store's virtual queue can also be an input or foot traffic signal. Furthermore, the number of visitors that register for contact tracing can also be used as an input or foot traffic signal. It has been discovered that techniques for providing access to scarce resources such as concert tickets can be applied to shopping appointments or queues, including a virtual queue, reservations, ticket balloting, pre-sales, and the like.

In the client application 320, displayed wait times can be organized or broken down by store type or other functional grouping. For example, the client application 320 can include an “Essentials” module that summarizes information about essential service providers such as grocery stores, pharmacies, clinics, banks, and other essential service providers. The client application 320 also permits the user to like or favourite or pin certain establishments to a frequently accessed or “Favourites” list or module.

The client application 320 permits dynamic updating of calculated and/or displayed wait times. For example, during travel to a store, a user may receive a notification that the wait time at the store has changed based on changing foot traffic signals such as a new input from another user.

The client application 320 can present the user with a dashboard of information including the predicted wait time for a current or future time or date, as well as additional information such as previously reported wait times, as well as useful marketing messages, such as tips, suggestions, incentives and messages from the store or others.

According to particular embodiments, the client application 320 can include a module that provides a questionnaire to allows users report foot traffic signals such as queue length, queue time, store hours, capacity limits, and so on. The client application 320 can include a notification seeking input or confirmation from a user on a periodic basis or upon detecting that the user's device location services reports that the device is within the geography of a store of interest. The client application 320 can include a module that provides points, rewards or other incentives for providing or confirming information.

### Shopping Appointments

According to particular embodiments, client application 320 provides the user with functionality to reserve or book a shopping appointment with a store or other establishment. According to these embodiments, the client application 320 is provided with an in-store planner module. Selecting a store opens a modal screen displaying the next 30 days with available shopping appointment slots (e.g., 9 am to 10 pm or as the case may be). The shopping appointments can be customized by retail users using the client application 320 (e.g., increments of 5 minutes, maximum of 40 appointments at a time, and a maximum appointment time of 90 minutes). A customer using the client application 320 can specify an appointment duration and the client application can be used during the appointment as a timer to show how much time is left on their appointment. Alternatively, a notification on the user device can advise the consumer how much time is left on their appointment.

According to particular embodiments, the server 130 or client application 320 can generate a calendar appointment that can be stored on client device 110 (e.g., in the native calendar application). A unique QR code is also generated that can be displayed on the user device and presented to a reader at the store (e.g., to facilitate check in or appointment verification). A QR code (quick response code) is a machine-readable optical label that contains information about the item to which it is attached. QR codes often contain data for a locator, identifier, or tracker that points to a website or application.

According to particular embodiments, the client application 320 can be used to create multiple appointments at different stores. The client application 320 can also be used to create a wait list for a preferred time (e.g., if all appointments are shown as occupied).

Furthermore, examples of the client application 320 can provide wait time prediction on checkout at the store, including during a shopping appointment. The checkout time prediction is based on signals such as place in queue, number of staff, number of self-checkout kiosks, average time other users spend in the store, predicted number of items in shopping baskets based on the amount of time spent in the store, probability of using coupons, time spent interacting with staff, and other metrics.

### Curb-Side Pickup and Queueing Services

According to particular embodiments, client application 320 provides the user with functionality to make and complete curb-side orders with a store or other establishment, and to manage queues for pickup and shopping appointments. According to these embodiments, the client application 320 is provided with a curb-side pickup module and/or a virtual queue module. Selecting a store opens a modal screen displaying an order form including a schedule of available curb-side pickup appointments and/or a virtual queue. For curb-side pickup functionality, after selection of an available appointment, the client application 320 provides an interface to select products or services to be fulfilled or provided. The client application 320 also provides a retail interface for retail users to approve, reject or modify orders and mark them as completed. The client application 320 can also provide reminders, assist with rescheduling of pickup appointments and verify orders closer to the pickup appointment, avoiding or reducing the possibility of an abandoned order. For virtual queue functionality, after selection of a store and a wait list, the client application 320 provides an interface to display the user's progress in the virtual queue, allowing the user to appear on-site at or near their appointed time rather than have to wait in the physical proximity of others in a physical queue.

According to particular embodiments, once an order is approved, the client application 320 can generate a calendar appointment that can be stored on the client device 110 (e.g., in the native calendar application). A unique QR code is also generated that can be displayed on the user device and presented to a reader at the store (e.g., to facilitate check in or appointment verification).

Furthermore, according to particular embodiments, once an order is approved, the server 130 or client application 320 can update a store's inventory system (shown as a third party system 150). Once the inventory is exhausted, the item can be removed from the list of available items either temporarily or permanently. The inventory system can also be populated by a supplier uploading an invoice using the client application 320.

The client application 320 offers functionality during the curb-side pickup process. The client application 320 can provide a “check-in” functionality that alerts the store of the user's arrival or approach to the designated pickup location. A map in the client application 320 can assist in locating the precise location. The store user can be notified upon “check-in”, provide an overview of the items to be fulfilled, and a location for the curb-side delivery. Upon meeting the customer, the client application 320 can provide functionality for scanning of the QR code to verify the order. Upon scanning of the QR code, the order can be automatically marked as completed.

Measurable foot traffic signals associated with the curb-side pickup experience, such as store traffic, the number of orders, the number of staff on-hand, time of day, and area traffic can be synthesized by the server 130 to provide the client application 320 associated with a waiting customer with an estimated time to fulfillment of the order, to provide a guided experience for the user.

Advantageously, use of the methods and systems disclosed herein can reduce the number of communications between store and customer, and can nudge the customer not to abandon an order, or if an order is abandoned, can alert the store that an order has or may become abandoned.

### Contact Tracing

According to particular embodiments, client application 320 provides the user with functionality to make appointments at hospitality venues and to provide contact details securely to the server 130. Selecting a venue opens a modal screen displaying a list of available appointments. After selection of an available appointment, the client application 320 has an interface to provide contact details for one or more of the parties. The client application 320 also provides a hospitality interface for hospitality users to approve, reject or modify appointments and mark them as completed.

During a contact tracing exercise, the server 130 can be queried for a list of all parties that were in attendance at the venue during the time and location of interest. Contact information through the system can be provided to local authorities or a secure message can be sent to the contacts encouraging them to be tested and/or to shelter in place to prevent further spread of the infection and/or to otherwise follow guidance from local health authorities. In some embodiments, all parties in a group may be required to provide contact information so that contact tracing is not only limited to persons that are carrying a client device 110. The server 130 can be configured to keep contact information for a limited duration, such as 30 days, or some other interval, to ensure that contacts can be traced without keeping records indefinitely.

Advantageously, use of the methods and systems herein can provide assurance to users that a venue is compliant with contact tracing efforts and can improve the guest experience for users by offering higher degree of safety and security for patrons.

The specification is directed to a system or platform for predictive, real-time foot traffic and contact tracing analytics.

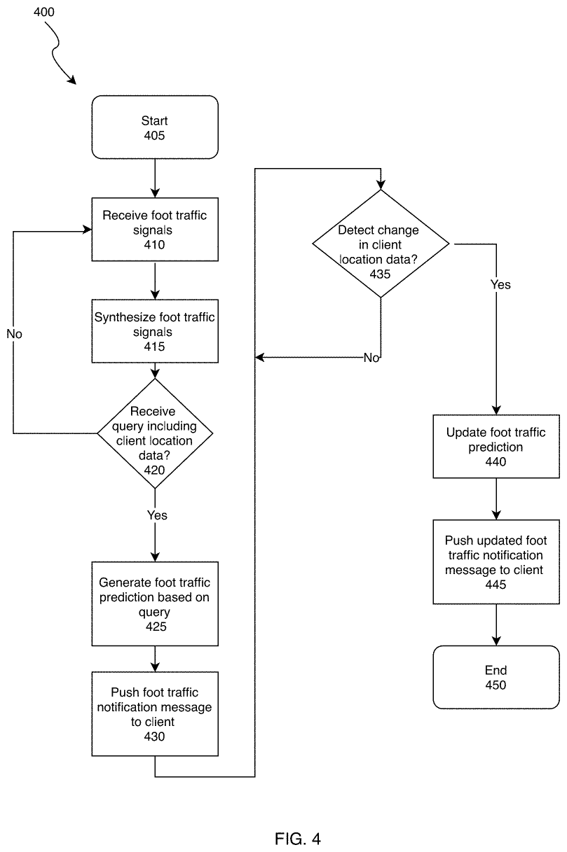

Flowcharts illustrating examples of generating and sending traffic signal notifications are shown in FIG. 4. These operations or methods can be carried out by applications or software executed by, for example, the processor of the server 130. The method can contain additional or fewer processes than shown and/or described, and can be performed in a different order. Computer-readable code executable by at least one of the processors to perform the method can be stored in a computer-readable storage medium, such as a non-transitory computer-readable medium.

With reference to FIG. 4, a method 400 starts at 405 and, at 410, the server 130 receives foot traffic signals from data store 140, client device 110 or third party system 150. At 415, server 130 synthesizes the received foot traffic signals. At 420, server 130 receives a data query from a client device 110 including a location data parameter from the client device 110. Responsive to the query, server 130 generates a foot traffic prediction at 425 and delivers or pushes a foot traffic notification message to the client device 110. Upon detecting a change in the location data parameter of the client device 110 at 435, server 130 updates the foot traffic prediction at 440 and delivers or pushes an updated foot traffic notification message to the client device 110 (e.g., for display). At 450, the method is completed. The method returns to receive and synthesize additional foot traffic signals at 410 and/or detect further changes in a location data parameter associated with the client device 110, providing continuing feedback to the client device 110.

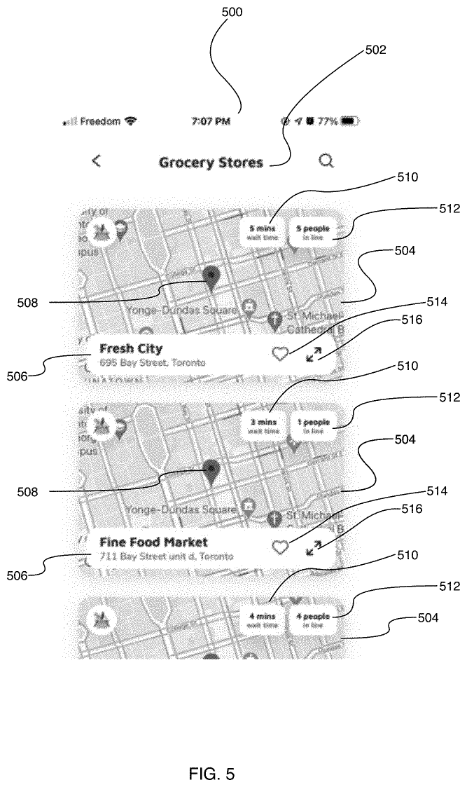

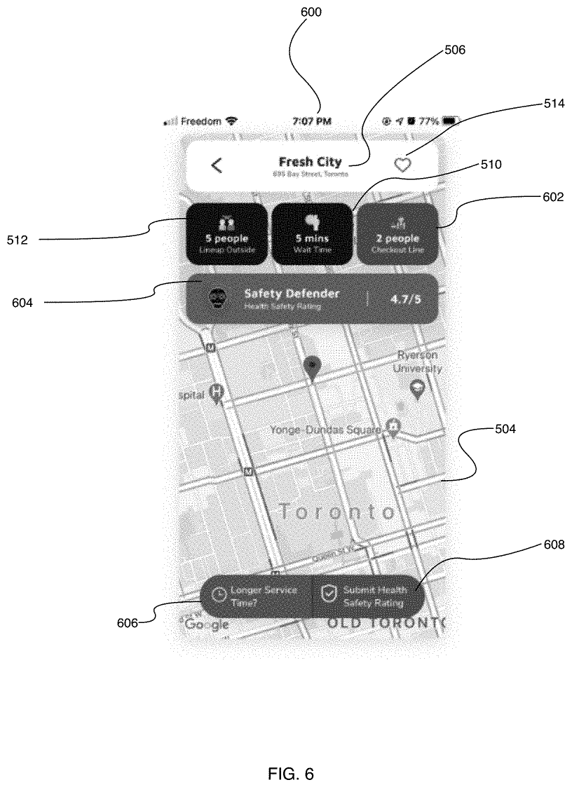

Examples of screenshots on the display of the client device 110 when loaded with client application 320 to operate in accordance with the present specification are depicted in FIG. 5 and FIG. 6 and described with continued reference to FIG. 4.

With reference to FIG. 5, screenshot 500 may be launched by accessing an application loaded on the client device 110. In one example, the application may require user authentication to proceed further. A map session is shown including store category 502, store cards 504, store labels 506, and store pins 508. User interface components are also shown including wait time display at location 510, queue length display at location 512, like button at location 514, and expand button at location 516. In FIG. 6, screenshot 600 may be launched by detecting a touch event at location 516 associated with the expand function. Screenshot 600 provides additional user interface components including a store checkout length display at location 602, a store score at location 604, and a feedback component including a location 606 associated with a queue feedback function and a location 608 associated with a safety feedback function. If the client application 320 detects a touch event at location 606 or location 608, then the client application 320 can display a modal window soliciting user input (not shown). The client application 320 is configured to send messages to the server 130 providing the feedback which can be used by the server 130 as foot traffic signals.

In particular embodiments, and as noted above, the client application 320 can display a virtual queue for a particular store (not shown). The virtual queue can be displayed on the same screen as wait times are displayed.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One general aspect includes a method in a server of maintaining a database of foot traffic signals related to a plurality of stores, receiving a plurality of foot traffic signals associated with the plurality of stores, synthesizing the plurality of foot traffic signals, and updating the database using the synthesized plurality of foot traffic signals. Upon receiving a data query from a user electronic device including a location data parameter, the server generates a foot traffic prediction based on the data query and the location data parameter from the database and forwards a foot traffic prediction notification for display on the user electronic device.

Implementations may include one or more of the following features: the foot traffic prediction includes a predicted wait time for selected of the plurality of stores within a proximity of the location data parameter; the plurality of foot traffic signals is associated with a plurality of factors and the synthesizing the plurality of foot traffic signals further comprises: applying a coefficient value to the plurality of factors for relative weighting of the plurality of factors; generating a provisional wait time; cross-referencing the provisional wait time to a time-series dataset using a weighted time-series algorithm to generate the predicted wait time; the foot traffic signals include one or more of the following data parameters: a time parameter, a date parameter, a weather parameter, a store size parameter, a store capacity parameter, a popularity parameter, a household income parameter, a historical data parameter, a data outlier parameter, a search engine parameter, and a virtual queue parameter; the foot traffic prediction notification can be dynamically updated based on a location service of the user electronic device and based on additional foot traffic signals received after forwarding the foot traffic prediction notification.

Another general aspect includes a method in a server of maintaining a database of appointments associated with some of the plurality of stores, receiving a calendar request from the user electronic device wherein the calendar request is a shopping appointment request on behalf of a consumer, updating the database and fulfilling the calendar request, and forwarding an appointment fulfillment confirmation notification for display on the user electronic device. The appointment fulfillment confirmation notification can include a first QR code to verify the shopping appointment request.

A further general aspect includes a method in a server of maintaining a database of orders associated with some of the plurality of stores; receiving an order request from the user electronic device wherein the order request is a curb-side order request on behalf of a consumer; updating the database and fulfilling the order request; and forwarding an order confirmation notification for display on the user electronic device. The order confirmation notification includes a second QR code to verify the curb-side order request.

Moreover, another general aspect includes a server including a processor, a memory, and a network interface device connected to a network, the memory storing instructions that, when executed by at least one processor, cause the at least one processor to: maintain a database of foot traffic signals related to a plurality of stores, receive a plurality of foot traffic signals associated with the plurality of stores, responsive to the receiving, synthesize the plurality of foot traffic signals, update the database using the synthesized plurality of foot traffic signals, receive a data query from a user electronic device including a location data parameter, generate a foot traffic prediction based on the data query and the location data parameter from the database, and forward a foot traffic prediction notification for display on the user electronic device.

Furthermore, another general aspect includes at least one non-transitory computer-readable storage medium storing instructions that, when executed by at least one processor, cause the at least one processor to maintain a database of foot traffic signals related to a plurality of stores, receive a plurality of foot traffic signals associated with the plurality of stores, responsive to the receiving, synthesize the plurality of foot traffic signals, update the database using the synthesized plurality of foot traffic signals, receive a data query from a user electronic device including a location data parameter, generate a foot traffic prediction based on the data query and the location data parameter from the database, and forward a foot traffic prediction notification for display on the user electronic device.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method comprising the steps of:
at a server comprising a processor, a memory, and a network interface device connected to a network,
maintaining a database of foot traffic signals related to a plurality of stores;
receiving a plurality of foot traffic signals associated with the plurality of stores;
responsive to the receiving, synthesizing the plurality of foot traffic signals;
updating the database using the synthesized plurality of foot traffic signals;
receiving a data query from a user electronic device including a location data parameter;
generating a foot traffic prediction based on the data query and the location data parameter from the database; and
forwarding a foot traffic prediction notification for display on the user electronic device.

2. The method of claim 1 wherein the foot traffic prediction comprises a predicted wait time for selected of the plurality of stores within a proximity of the location data parameter.

3. The method of claim 1 wherein the plurality of foot traffic signals is associated with a plurality of factors and the synthesizing the plurality of foot traffic signals further comprises: applying a coefficient value to the plurality of factors for relative weighting of the plurality of factors; generating a provisional wait time; and cross-referencing the provisional wait time to a time-series dataset using a weighted time-series algorithm to generate a predicted wait time.

4. The method of claim 1 wherein the foot traffic signals comprise one or more of the following data parameters: a time parameter, a date parameter, a weather parameter, a store size parameter, a store capacity parameter, a popularity parameter, a household income parameter, a historical data parameter, a data outlier parameter, a search engine parameter, and a virtual queue parameter.

5. The method of claim 1 further comprising:
at the server, maintaining a second database of appointments associated with some of the plurality of stores;
receiving a calendar request from the user electronic device wherein the calendar request is a shopping appointment request on behalf of a consumer;
updating the second database and fulfilling the calendar request; and
forwarding an appointment fulfillment confirmation notification for display on the user electronic device.

6. The method of claim 5 wherein the appointment fulfillment confirmation notification includes a first QR code to verify the shopping appointment request.

7. The method of claim 5 further comprising:
at the server, maintaining a third database of orders associated with some of the plurality of stores;
receiving an order request from the user electronic device wherein the order request is a curb-side order request on behalf of a consumer;
updating the third database and fulfilling the order request; and
forwarding an order confirmation notification for display on the user electronic device.

8. The method of claim 7 wherein the order confirmation notification includes a second QR code to verify the curb-side order request.

9. The method of claim 1 wherein the foot traffic prediction notification is dynamically updated based on a location service of the user electronic device and based on additional foot traffic signals received after forwarding the foot traffic prediction notification.

10. A server comprising a processor, a memory, and a network interface device connected to a network, the memory storing instructions that, when executed by at least one processor, cause the at least one processor to:
maintain a database of foot traffic signals related to a plurality of stores;
receive a plurality of foot traffic signals associated with the plurality of stores;
responsive to the receiving, synthesize the plurality of foot traffic signals;
update the database using the synthesized plurality of foot traffic signals;
receive a data query from a user electronic device including a location data parameter;
generate a foot traffic prediction based on the data query and the location data parameter from the database; and
forward a foot traffic prediction notification for display on the user electronic device.

11. At least one non-transitory computer-readable storage medium storing instructions that, when executed by at least one processor, cause the at least one processor to:
maintain a database of foot traffic signals related to a plurality of stores;
receive a plurality of foot traffic signals associated with the plurality of stores;
responsive to the receiving, synthesize the plurality of foot traffic signals;
update the database using the synthesized plurality of foot traffic signals;
receive a data query from a user electronic device including a location data parameter;
generate a foot traffic prediction based on the data query and the location data parameter from the database; and
forward a foot traffic prediction notification for display on the user electronic device.
